(19) United States
(12) Patent Application Publication
(10) Pub. No.: US 2026/0014327 A1
(43) Pub. Date: Jan. 15, 2026

(54) METHOD AND SYSTEM FOR MANAGING PATH OF NEEDLE BY USING DIGITAL TWIN, AND NON-TRANSITORY COMPUTER-READABLE RECORDING MEDIUM

(71) Applicant: RIMSCIENCE CO., LTD., Seoul (KR)

(72) Inventor: Sang Jin YOON, Incheon (KR)

(73) Assignee: RIMSCIENCE CO., LTD., Seoul (KR)

(21) Appl. No.: 18/846,063

(22) PCT Filed: Mar. 10, 2023
(86) PCT No.: PCT/KR2023/003326

§ 371 Date: Mar. 12, 2025

(30) Foreign Application Priority Data
Mar. 11, 2022 (KR) 10-2022-0030989
Feb. 16, 2023 (KR) 10-2023-0021076

(51) Int. Cl. A61M 5/42; G16H 40/63

(52) U.S. Cl. CPC A61M 5/427; G16H 40/63; A61M 2205/3375

(57) ABSTRACT

According to one aspect of the present invention, provided is a method for managing the path of a needle by using a digital twin, comprising the steps of: referring to a first angle at which a probe of an ultrasound device makes contact with a first point of the body of a patient, so as to determine a digital twin model corresponding to the body of the patient; and referring to the digital twin model so as to determine the angle at which a medical needle of a needle control device will enter the first point or a second point of the body of the patient.

### FIELD OF THE INVENTION

The present invention relates to a method, system, and non-transitory computer-readable recording medium for managing a needle path using a digital twin.

### BACKGROUND

Methods such as injecting a solution into an epidural space inside ligamenta flava for regional anesthesia are widely used in painless delivery in obstetrics, surgical procedures, and the like.

The epidural space is located between the ligamenta flava and a dural sac with a depth of only about 2 to 8 mm, requiring precise operations. For example, if a needle does not reach the epidural space during the course of needle insertion, it may cause a problem with the administration of the solution, and if the needle pierces through the epidural space, it may cause nerve damage due to cerebrospinal fluid leakage (e.g., dural sac injury). Therefore, it is crucial to accurately position the needle in the epidural space.

However, according to the techniques introduced so far as well as the conventional techniques, medical needles have been inserted primarily relying on perception of physicians, which causes a problem that the results are affected by skill levels, condition, fatigue levels, and the like of the physicians. Further, a method for inserting medical needles using air pressure has been introduced, which has a problem that the accuracy varies depending on physical condition, ages, and the like of patients.

In this connection, the inventor(s) present a novel and inventive technique capable of accurately positioning a medical needle at a desired target point with reference to a digital twin model corresponding to a patient's body.

### SUMMARY OF THE INVENTION

One object of the present invention is to solve all the above-described problems in the prior art.

Another object of the invention is to accurately position a medical needle at a desired target point (e.g., epidural space) inside a patient's body with reference to a digital twin model corresponding to the patient's body.

The representative configurations of the invention to achieve the above objects are described below.

According to one aspect of the invention, there is provided a method for managing a needle path using a digital twin, the method comprising the steps of: determining a digital twin model corresponding to a body of a patient with reference to a first angle at which a probe of an ultrasound apparatus makes contact with a first point of the body of the patient; and determining an angle at which a medical needle of a needle control apparatus penetrates from the first point or a second point of the body of the patient with reference to the digital twin model.

According to another aspect of the invention, there is provided a system for managing a needle path using a digital twin, the system comprising: a model determination unit configured to determine a digital twin model corresponding to a body of a patient with reference to a first angle at which a probe of an ultrasound apparatus makes contact with a first point of the body of the patient; and an angle determination unit configured to determine an angle at which a medical needle of a needle control apparatus penetrates from the first point or a second point of the body of the patient with reference to the digital twin model.

In addition, there are further provided other methods and systems to implement the invention, as well as non-transitory computer-readable recording media having stored thereon computer programs for executing the methods.

According to the invention, it is possible to accurately position a medical needle at a desired target point (e.g., epidural space) inside a patient's body with reference to a digital twin model corresponding to the patient's body.

### BRIEF DESCRIPTION OF THE DRAWINGS

FIG. 1 schematically shows the configuration of an entire system for managing a needle path according to one embodiment of the invention.

FIG. 2 specifically shows the internal configuration of a needle path management system according to one embodiment of the invention.

FIG. 3 illustratively shows a needle control apparatus according to one embodiment of the invention.

### DETAILED DESCRIPTION OF THE PREFERRED EMBODIMENTS

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the invention will be described in detail with reference to the accompanying drawings to enable those skilled in the art to easily implement the invention.

### Configuration of the Entire System

FIG. 1 schematically shows the configuration of the entire system for managing a needle path according to one embodiment of the invention.

As shown in FIG. 1, the entire system according to one embodiment of the invention may comprise a communication network 100, a needle path management system 200, a needle control apparatus 300, and an ultrasound apparatus 400.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may be the Internet or the World Wide Web (WWW). However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, WiFi-Direct communication, Long Term Evolution (LTE) communication, 5G communication, Bluetooth communication (including Bluetooth Low Energy (BLE) communication), infrared communication, and ultrasonic communication.

Next, the needle path management system 200 according to one embodiment of the invention may communicate with the needle control apparatus 300 and the ultrasound apparatus 400 to be described below via the communication network 100, and may function to determine a digital twin model corresponding to a body of a patient with reference to a first angle at which a probe (not shown) of the ultrasound apparatus 400 makes contact with a first point of the body of the patient, and determine an angle at which a medical needle 310 of the needle control apparatus 300 penetrates from the first point or a second point of the body of the patient with reference to the digital twin model. Meanwhile, the needle path management system 200 may be digital equipment having a memory means and a microprocessor for computing capabilities, and may be, for example, a server system operating on the communication network 100. The configuration and functions of the needle path management system 200 according to one embodiment of the invention will be described below.

Next, the needle control apparatus 300 according to one embodiment of the invention is digital equipment capable of connecting to and then communicating with the needle path management system 200, and may comprise a medical needle 310, an actuator 320, and an operation property determination unit 330 (see FIG. 3). Specifically, the needle control apparatus 300 according to one embodiment of the invention may function to cause the medical needle 310 to penetrate into a patient's body as the actuator 320 operates on the basis of operation properties (e.g., a direction, speed, and acceleration) determined by the operation property determination unit 330.

Here, the medical needle 310 may include a sensor module (not shown) for acquiring sensing information associated with the patient's body tissue through which the needle passes, or may be connected (e.g., electrically connected) to such a sensor module.

For example, the medical needle 310 may include at least one sensor module (not shown) among a force sensor module, a torque sensor module, a pressure sensor module, a displacement sensor module, an ultrasonic sensor module, an optical sensor module (e.g., a Raman spectroscopic optical sensor module), a temperature sensor module, an acidity sensor module, a gas pressure sensor module, an electromagnetic wave sensor module, and a chemical substance detection sensor module, and information on force (e.g., reaction force, torque, or pressure specified by elasticity or density of the body tissue) exerted on the medical needle 310 by the body tissue through which one end (e.g., a tip) of the medical needle 310 passes, information on temperature, acidity, gas pressure, electromagnetic properties (e.g., responses according to ultra-high frequency waves such as microwaves or millimeter waves), bodily fluid properties (e.g., physicochemical properties of lymph fluid, intestinal fluid (e.g., fluid inside a mouth, esophagus, stomach, small intestine, or large intestine), pleural fluid, abdominal fluid, urine components, fecal components, or serous exudate), or blood properties (e.g., physicochemical properties such as oxygen saturation, blood flow distribution, or blood sugar) of the body tissue through which the one end of the medical needle 310 passes, and the like may be acquired on the basis of the at least one sensor module.

Further, the medical needle 310 may include a gravity sensor (hereinafter, “G-sensor”). The G-sensor included in the medical needle 310 may be used to specify an angle of the medical needle 310 (e.g., a second angle to be described below). Furthermore, the G-sensor included in the medical needle 310 may also be used to specify a relative position or direction of progression of the medical needle 310 with respect to a digital twin model corresponding to the patient's body.

Meanwhile, it is noted that the configuration and functions of the needle control apparatus 300 according to one embodiment of the invention are not limited to those described above, and may be changed or added with reference to the disclosures of Korean Registered Patent No. 10-2363626 (which are deemed to be incorporated herein in their entirety).

Next, the ultrasound apparatus 400 according to one embodiment of the present invention is digital equipment capable of connecting to and then communicating with the needle path management system 200, and may include a probe. Specifically, the ultrasound apparatus 400 according to one embodiment of the invention may function to acquire ultrasound images of the patient's body by contacting the probe to the patient's body. Here, the probe may include a G-sensor (which may be a sensor that is separate from the G-sensor included in the medical needle 310 of the needle control apparatus 300 but has the same function). The G-sensor included in the probe may be used to specify an angle of the probe (e.g., a first angle to be described below).

### Configuration of the Needle Path Management System

Hereinafter, the internal configuration of the needle path management system 200 crucial for implementing the invention and the functions of the respective components thereof will be discussed.

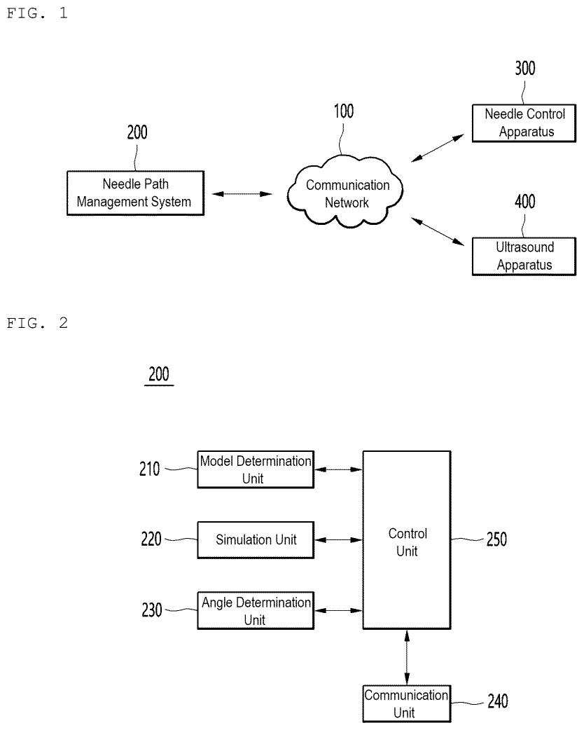

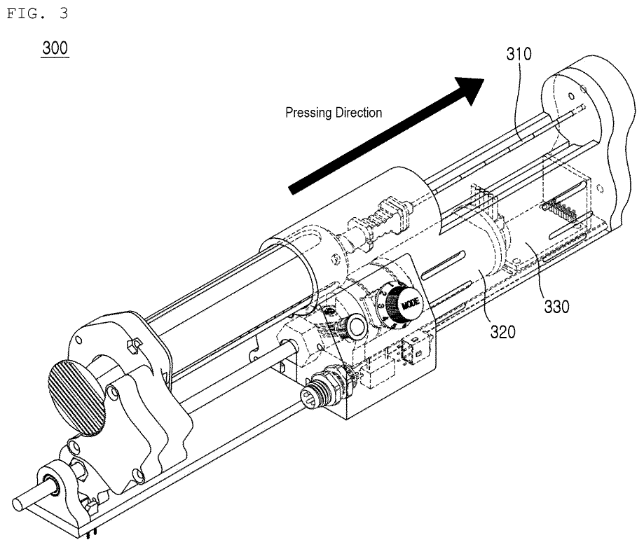

FIG. 2 specifically shows the internal configuration of the needle path management system 200 according to one embodiment of the invention.

As shown in FIG. 2, the needle path management system 200 according to one embodiment of the invention may comprise a model determination unit 210, a simulation unit 220, an angle determination unit 230, a communication unit 240, and a control unit 250. According to one embodiment of the invention, at least some of the model determination unit 210, the simulation unit 220, the angle determination unit 230, the communication unit 240, and the control unit 250 of the needle path management system 200 may be program modules to communicate with an external system (not shown). The program modules may be included in the needle path management system 200 in the form of operating systems, application program modules, or other program modules, while they may be physically stored in a variety of commonly known storage devices. Further, the program modules may also be stored in a remote storage device that may communicate with the needle path management system 200. Meanwhile, such program modules may include, but are not limited to, routines, subroutines, programs, objects, components, data structures, and the like for performing specific tasks or executing specific abstract data types as will be described below in accordance with the invention.

Meanwhile, the above description is illustrative although the needle path management system 200 has been described as above, and it will be apparent to those skilled in the art that at least a part of the components or functions of the needle path management system 200 may be implemented in the needle control apparatus 300 or the ultrasound apparatus 400 or included in an external system (not shown), as necessary.

First, the model determination unit 210 according to one embodiment of the invention may determine a digital twin model corresponding to a body of a patient with reference to a first angle at which a probe of the ultrasound apparatus 400 makes contact with a first point of the body of the patient. Here, the first point may refer to any one point located on the skin of the patient's body. Further, the first angle may refer to an angle formed between the contact surface of the probe and a reference plane (e.g., the ground surface) when the probe is in contact with the first point of the patient's body. According to one embodiment of the invention, the first angle may be specified on the basis of a reference coordinate system of the probe (more specifically, a reference coordinate system of the G-sensor of the probe).

For example, the model determination unit 210 according to one embodiment of the invention may specify the first angle in response to an operator (e.g., medical personnel) of the ultrasound apparatus 400 contacting the probe to the first point of the patient's body, and may determine (or generate) a digital twin model corresponding to the patient's body with reference to the specified first angle. More specifically, the model determination unit 210 according to one embodiment of the invention may determine (or generate) a digital twin model corresponding to the patient's body within a predetermined range or radius from the first point with respect to the specified first angle. Here, the range or radius in which the digital twin model is generated may be dynamically determined, but may be determined to at least include a target point (e.g., epidural space) inside the patient's body.

Meanwhile, the model determination unit 210 according to one embodiment of the invention may generate the digital twin model on the basis of ultrasound images acquired from the ultrasound apparatus 400 or image information acquired from medical imaging equipment other than the ultrasound apparatus 400 (e.g., CT (Computer Tomography) or MRI (Magnetic Resonance Imaging) equipment). For example, the model determination unit 210 according to one embodiment of the invention may specify anatomical information of the patient (e.g., information on locations and shapes of organs, tissues, skeletons, and the like) from the ultrasound images acquired from the ultrasound apparatus 400 or the image information acquired from the medical imaging equipment, and may generate the digital twin model by modeling the specified anatomical information in three dimensions. However, it is noted that the manner in which the model determination unit 210 generates the digital twin model is not limited to those mentioned above, but may be diversely changed as long as the objects of the invention may be achieved.

Next, the simulation unit 220 according to one embodiment of the invention may function to simulate a path in which the medical needle 310 of the needle control apparatus 300 travels from a predetermined point of the patient's body to a target point with respect to the digital twin model determined as above.

For example, the simulation unit 220 according to one embodiment of the invention may simulate the path in which the medical needle 310 travels in response to an operator of the needle control apparatus 300 (who may be the same as or different from the operator of the ultrasound apparatus 400) causing an actual medical needle or a virtual medical needle to penetrate from the first point of the digital twin model (or a second point located within a predetermined range of the first point) (here, the second point may refer to any one point located on the skin of the patient's body, like the first point) at a second angle. Here, simulating the path in which the medical needle travels may mean simulating whether the medical needle reaches the target point and the degree of damage to the body during that course is not greater than a predetermined level when the medical needle penetrates into the patient's body along the path. If a result of the simulation according to the path in which the medical needle penetrates from the first point (or the second point) at the second angle does not meet the above criteria (i.e., the medical needle reaches the target point and the degree of damage to the body during that course is not greater than the predetermined level), the operator of the needle control apparatus 300 may cause the medical needle to penetrate from the first point (or the second point) of the digital twin model at an angle different from the second angle (i.e., the simulation may be reperformed). Meanwhile, according to one embodiment of the invention, the actual medical needle refers to the medical needle 310 of the needle control apparatus 300 and may be applied to the digital twin model augmented in a real-world environment during the course of the simulation. Further, the virtual medical needle is a virtualization of the medical needle 310 of the needle control apparatus 300 and may be applied to the digital twin model presented in a virtual environment (e.g., shown on a display) during the course of the simulation.

Meanwhile, before performing the simulation, the simulation unit 220 according to one embodiment of the invention may synchronize a reference coordinate system of the medical needle (i.e., the actual or virtual medical needle) (more specifically, a reference coordinate system of the G-sensor of the medical needle) with the reference coordinate system of the probe (e.g., to specify the matching or relationship between the two reference coordinate systems, or to perform a transformation into a predetermined reference coordinate system). The simulation unit 220 according to one embodiment of the invention may guide the medical needle to be positioned at the first point of the digital twin model in a state in which the two coordinate systems are synchronized as above (e.g., an alarm may be generated when the tip of the medical needle is positioned at the first point), and the simulation may be initiated when the medical needle penetrates from the first point (or the second point) according to the guidance.

Meanwhile, according to one embodiment of the invention, the second angle may refer to an angle formed between the medical needle and a reference plane (e.g., the ground surface) when the medical needle (i.e., the actual or virtual medical needle) penetrates from the first point of the digital twin model. According to one embodiment of the invention, the second angle may be specified on the basis of the reference coordinate system of the medical needle (more specifically, the reference coordinate system of the G-sensor of the medical needle), and may also be specified on the basis of the reference coordinate system of the probe or a predetermined reference coordinate system as the reference coordinate system of the medical needle is synchronized with the reference coordinate system of the probe as described above.

Next, the angle determination unit 230 according to one embodiment of the invention may determine an angle at which the medical needle 310 of the needle control apparatus 300 penetrates from the first point (or the second point) of the patient's body with reference to the digital twin model (specifically, the result of the simulation performed with respect to the digital twin model).

Specifically, if it is simulated that when the medical needle (i.e., the actual or virtual medical needle) follows the simulated path (i.e., the path in which the medical needle penetrates from the first point (or the second point) at the second angle), the medical needle reaches the target point and the degree of damage to the body during that course is not greater than a predetermined level, the second angle may be determined as an angle at which the medical needle 310 of the needle control apparatus 300 penetrates from the first point (or the second point) of the patient's body, i.e., a penetration angle.

Further, when the penetration angle is determined as described above, the angle determination unit 230 may cause the medical needle 310 of the needle control apparatus 300 to be inserted from the first point (or the second point) of the patient's body according to the determined angle.

For example, the angle determination unit 230 according to one embodiment of the invention may transmit a trigger signal to the needle control apparatus 300 in response to the medical needle 310 of the needle control apparatus 300 being positioned at the first point (or the second point) at the penetration angle (i.e., the second angle). Here, the trigger signal may be a signal that allows the medical needle 310 of the needle control apparatus 300 to penetrate into the patient's body (or a signal that allows the operator of the needle control apparatus 300 to cause the medical needle 310 of the needle control apparatus 300 to penetrate into the patient's body). According to one embodiment of the invention, the medical needle 310 of the needle control apparatus 300 is inserted (or penetrates) into the patient's body according to the trigger signal, and travels along the same path as the simulated path to the target point after being inserted into the patient's body.

Thus, according to the invention, the medical needle 310 of the needle control apparatus 300 may accurately reach the target point while minimizing damage to the patient's body tissue.

Meanwhile, the angle determination unit 230 according to one embodiment of the invention may determine the angle at which the medical needle 310 of the needle control apparatus 300 penetrates from the first point (or the second point) of the patient's body, referring only to the digital twin model without referring to the simulation result.

For example, even if the above simulation is not performed by the simulation unit 220, the angle determination unit 230 according to one embodiment of the invention may determine the angle at which the medical needle 310 of the needle control apparatus 300 penetrates from the first point (or the second point) of the patient's body, using (a prediction result of) a predetermined artificial intelligence-based model (e.g., a model that is trained to, when the digital twin model and the first point (or the second point) are inputted as input data, output the corresponding penetration angle). Meanwhile, the angle determination unit 230 may determine the angle at which the medical needle 310 of the needle control apparatus 300 penetrates from the first point (or the second point) of the patient's body with reference to a lookup table (not shown) for the penetration angle corresponding to the digital twin model and the first point (or the second point).

Next, the communication unit 240 according to one embodiment of the invention may function to enable data transmission/reception from/to the model determination unit 210, the simulation unit 220, and the angle determination unit 230.

Lastly, the control unit 250 according to one embodiment of the invention may function to control data flow among the model determination unit 210, the simulation unit 220, the angle determination unit 230, and the communication unit 240. That is, the control unit 250 according to the invention may control data flow into/out of the needle path management system 200 or data flow among the respective components of the needle path management system 200, such that the model determination unit 210, the simulation unit 220, the angle determination unit 230, and the communication unit 240 may carry out their particular functions, respectively.

The embodiments according to the invention as described above may be implemented in the form of program instructions that can be executed by various computer components, and may be stored on a computer-readable recording medium. The computer-readable recording medium may include program instructions, data files, and data structures, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler, but also high-level language codes that can be executed by a computer using an interpreter. The above hardware devices may be changed to one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for managing a needle path using a digital twin, the method comprising the steps of:
determining a digital twin model corresponding to a body of a patient with reference to a first angle at which a probe of an ultrasound apparatus makes contact with a first point of the body of the patient; and
determining an angle at which a medical needle of a needle control apparatus penetrates from the first point or a second point of the body of the patient with reference to the digital twin model.

2. The method of claim 1, wherein in response to the penetration angle being determined, the medical needle is inserted from the first point or the second point of the body of the patient according to the determined angle.

3. The method of claim 1, further comprising the step of:
before the angle determination step, simulating a path in which the medical needle of the needle control apparatus travels from the first point or the second point of the body of the patient to a target point with respect to the digital twin model.

4. The method of claim 3, wherein in the simulation step, a reference coordinate system of the medical needle is synchronized with a reference coordinate system of the probe.

5. The method of claim 3, wherein in the angle determination step, the angle at which the medical needle penetrates from the first point or the second point of the body of the patient is determined with reference to a result of the simulation.

6. The method of claim 1, wherein the medical needle and the probe include gravity sensors.

7. A non-transitory computer-readable recording medium having stored thereon a computer program for executing the method of claim 1.

8. A system for managing a needle path using a digital twin, the system comprising:
a model determination unit configured to determine a digital twin model corresponding to a body of a patient with reference to a first angle at which a probe of an ultrasound apparatus makes contact with a first point of the body of the patient; and
an angle determination unit configured to determine an angle at which a medical needle of a needle control apparatus penetrates from the first point or a second point of the body of the patient with reference to the digital twin model.

9. The system of claim 8, wherein the angle determination unit is configured to, in response to the penetration angle being determined, cause the medical needle to be inserted from the first point or the second point of the body of the patient according to the determined angle.

10. The system of claim 8, further comprising:
a simulation unit configured to simulate a path in which the medical needle of the needle control apparatus travels from the first point or the second point of the body of the patient to a target point with respect to the digital twin model.

11. The system of claim 10, wherein the simulation unit is configured to synchronize a reference coordinate system of the medical needle with a reference coordinate system of the probe.

12. The system of claim 10, wherein the angle determination unit is configured to determine the angle at which the medical needle penetrates from the first point or the second point of the body of the patient with reference to a result of the simulation.

13. The system of claim 8, wherein the medical needle and the probe include gravity sensors.
